(19) United States
(12) Patent Application Publication
(10) Pub. No.: US 2024/0164768 A1
(43) Pub. Date: May 23, 2024

(54) ROBOTIC SYSTEMS FOR USE THROUGH A NATURAL ORIFICE AND SUTURING WITHIN THE GASTROINTESTINAL TRACT

(71) Applicant: Boston Scientific Scimed, Inc., Maple Grove, MN (US)

(72) Inventors: John Mims, Austin, TX (US); Luis Amador, JR., Pflugerville, TX (US); Jason Mehta, Austin, TX (US); Thomas Neudeck, Austin, TX (US); Michael Fries, Kingsland, TX (US); Vladimir Mitelberg, Austin, TX (US)

(73) Assignee: BOSTON SCIENTIFIC SCIMED, INC., Maple Grove, MN (US)

(21) Appl. No.: 18/515,150

(22) Filed: Nov. 20, 2023

Related U.S. Application Data
(60) Provisional application No. 63/427,054, filed on Nov. 21, 2022.

(51) Int. Cl. A61B 17/04; A61B 34/20

(52) U.S. Cl. CPC A61B 17/0469; A61B 34/20; A61B 2017/00818

(57) ABSTRACT

Flexible robotic systems are provided for guidance and to facilitate reliable suturing within the gastrointestinal tract. Such robotic systems provide assistance, assurance, and/or validation to compensate for the level of skill and experience of the operator, and to expedite completion of bariatric procedures approached from within the stomach.

### CROSS-REFERENCE TO RELATED APPLICATIONS

The present application claims the benefit of priority under 35 U.S.C. § 119 to U.S. Provisional Application No. 63/427,054, filed Nov. 21, 2022, the disclosure of which is hereby incorporated herein by reference in its entirety for all purposes. Any and all priority claims identified in the Application Data Sheet, or any correction thereto, are hereby incorporated by reference under 37 C.F.R. § 1.57.

This application is related to co-assigned U.S. Pat. Nos. 8,287,556, 9,867,610, 11,083,364, and 11,051,800, which are hereby incorporated by reference herein in their entireties.

### BACKGROUND OF THE INVENTION

### 1. Field of the Invention

The present disclosure relates to treatment systems for treating a mammalian body through a natural orifice. More particularly, this disclosure relates to robotic systems adapted to facilitate operation of such treatment systems and methods of treatment.

### 2. State of the Art

The condition of obesity means an individual has too much body fat and also that an individual's weight is higher than what is considered to be healthy for their height. Biology plays a big role in why some people become obese, but not getting enough exercise, eating more food than the body can use, and drinking too much alcohol also contributes to people becoming obese. Obesity is a major health threat because excess weight puts more stress on every part of the body and puts people at risk of several health problems, such as diabetes, heart disease, and stroke.

For some people, lifestyle changes like maintaining a healthy diet and exercising regularly can help them drop body fat and stop being obese. For others though, it can be extremely difficult to lose body fat and consistently maintain weight loss. Medications for losing weight are available on the market, but some can have serious side effects, may not actually be effective, and patient compliance for taking regular medication can be low. For obese individuals who cannot lower their amount of body fat through lifestyle changes or medications, various surgical options have become available.

Gastric bypass surgery was the first commonly practiced procedure performed to make the stomach smaller. The procedure involves stapling portions of the stomach wall together and then relocating a small part of the small intestine to the newly formed stomach pouch. By reducing the size of the stomach, the stomach holds less food, the individual obtains a sensation of fullness quicker, fewer calories are eaten, fewer calories are absorbed, and weight loss results. However, there are downsides to the procedure. The procedure is an open surgical procedure which has its own risks, including the potential for complications and infection, and can have an extensive post-surgical recovery period. The procedure is also relatively complicated requiring a reconfiguration of the small intestines. Also, over time the staples can release allowing the stomach to re-enlarge or the stomach volume can naturally enlarge over time, rendering the procedure less effective.

Another procedure is the ‘gastric banding’ procedure, primarily with the LAP-BAND® system, in which an inflatable band is inserted through the abdomen and about the stomach in a laparoscopic procedure. The band is wrapped around the upper part of the stomach to form a stoma, or ring. Attached to the ring is a thin tube leading to an access port that is implanted under the skin. A balloon attached to the band contacts the stomach and can be inflated (or deflated) with saline via the access port using a needle. Adding saline tightens the stoma about the stomach to cause an earlier sensation of satiety. If the band is too tight, saline can be withdrawn. An advantage of the gastric banding is that it can be performed in a minimally invasive manner with small laparoscopic incisions into the abdomen with consequent reduced recovery time, and that no reconfiguration of the small intestines is required. Nevertheless, the procedure still requires incisions, infection can result, and the recovery can be uncomfortable. In addition, the patient is left with a permanent port just under their skin which can be undesirable to some.

These types of procedures, when all goes well, can be effective, but as stated come with the risks associated with open or laparoscopic surgery, and for that reason they are only prescribed in cases of extreme obesity.

Incisionless fully endoscopic methods of reducing the capacity of the stomach have been developed to surgically treat obesity. Broadly, such methods endoluminally approximate tissue at a portion of the stomach, including at least a portion of the greater curvature of the stomach. The method includes making a pattern of endoscopic stitches in which a significant portion of the stomach is closed off. The resulting stomach reduction procedure can provide a seventy to seventy-five percent reduction in available stomach volume. Because the procedure is incisionless, it is safer to patients and offers an easier recovery.

While the procedure is shown to be an effective method to reduce the capacity of the stomach, create an earlier sensation of satiety, and effect weight loss in an obese patient, there remain obstacles to its application. Bariatric procedures have conventionally been practiced by bariatric surgeons who approach stomach reduction from outside the stomach and are not as familiar with surgery on gastrointestinal structures when viewed from inside the gastrointestinal tract. Meanwhile, gastroenterologists who are more familiar operating on the stomach from the interior do not have the familiarity with bariatric procedures and surgical suturing and as a consequence have a reduced comfort level with such surgeries.

### SUMMARY OF THE INVENTION

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

Flexible robotic systems are provided for guidance and to facilitate reliable suturing within the gastrointestinal tract. Such robotic systems provide assistance, assurance, and/or validation to compensate for the level of skill and experience of the operator, and to expedite completion of bariatric procedures approached from within the stomach.

In accordance with various principles of the present disclosure, a robotic suturing system is configured for use in a gastrointestinal tract of a human patient, the human patient having a natural orifice in communication with the gastrointestinal tract. In some aspects, the system includes a flexible, shapeable endoluminal robot adapted to be inserted into the gastrointestinal tract through the natural orifice, the endoluminal robot having a proximal end and a distal end; and a suturing tool removably coupled to the distal end of the flexible endoluminal robot, the suturing tool adapted to grasp tissue and advance a needle with suture through the grasped tissue; a robot control system configured to control movement of the endoluminal robot and the needle on the suturing tool; an imaging system for capturing real-time images of the gastrointestinal tract; and a display for presenting the real-time images to an operator.

In some aspects, the robot control system includes actuators to operate the endoluminal robot and the suturing tool, and an operator interface to control the actuators.

In some aspects, the system further includes sensors adapted to map at least a portion of the gastrointestinal tract.

In some aspects, the imaging system includes at least one of optical sensors, laser scanning sensors, distance sensors, ultrasound sensors, CT scan devices, and LIDAR sensors.

In some aspects, the system further includes a mapping system to define a virtual map of a portion of the gastrointestinal tract. In some aspects, the system further includes a modelling system to propose pre-operative planning of a procedure in a gastrointestinal tract. In some aspects, the modelling system is adapted to project aspects of the preoperative planning over the real-time images.

In some aspects, the system further includes at least one sensor adapted to determine whether the needle is inserted through a full thickness of the tissue.

In some aspects, the display is also adapted to present virtual images to guide the operator. In some aspects, the virtual images include proposed suturing locations. In some aspects, the virtual images include warning zones for non-suturing. In some aspects, the virtual images include proposed suturing locations. In some aspects, the virtual images include proposed suturing orientations. In some aspects, the virtual images include anatomical landmarks. In some aspects, the virtual images include fiducials.

In accordance with various principles of the present disclosure, a method of suturing tissue of a portion of a gastrointestinal tract of a patient includes mapping at least a portion of the gastrointestinal tract; obtaining imaging data of the patient; constructing a digital twin of the portion of the gastrointestinal tract; identifying proposed locations for suturing in the digital twin; providing a suturing system with an imaging system capable of obtaining real-time video images; inserting the suturing system into the gastrointestinal tract through a natural orifice; virtually overlaying the proposed locations over real-time video images; and suturing the portion of the gastrointestinal system.

In some aspects, the portion of the gastrointestinal tract is the stomach.

In some aspects, the suturing reshapes the stomach.

In some aspects, the suturing occurs at the proposed locations.

In some aspects, the method further includes updating the proposed locations for suturing as the portion of the gastrointestinal tract is sutured.

In some aspects, the method further includes providing virtual overlays of warning zones over real-time video images.

In some aspects, the method further includes updating the virtual overlays for warning zones as the gastrointestinal tract is sutured.

In some aspects, the method further includes providing overlays of an estimated current volume reduction of the portion of the gastrointestinal tract effected by the suturing.

In some aspects, the method further includes providing overlays of anatomical landmarks over real-time video images.

In some aspects, the method further includes providing fiducials on landmarks of the gastrointestinal tract to guide a human operator. In some aspects, the fiducials are virtual and overlaid on real-time images. In some aspects, the fiducials are physical. In some aspects, the fiducials can be sensed by sensors of the system.

In some aspects, the method further includes monitoring whether the needle passes through a full thickness of the tissue.

In some aspects, the method further includes determining whether the needle passes through a full thickness of tissue by using force sensors. In some aspects, the determining analyzes at least one signal from the force sensors to determine whether the needle passes through the full thickness of tissue.

In some aspects, the method further includes providing an insufflation gas into the gastrointestinal tract; and adjusting the amount of insufflation gas as the gastrointestinal space is sutured.

In some aspects, the method further includes before suturing, grasping tissue; monitoring a force on the grasped tissue; and releasing the grasped tissue if the force exceeds a threshold.

In some aspects, the needle is coupled to a suture that is detectable by the robotic system. In some aspects, the suture includes a surface texture. In some aspects, the suture includes a surface pattern.

In some aspects, the method further includes monitoring a tension on the suture.

In some aspects, the method further includes controlling a tension on the suture.

In accordance with various principles of the present disclosure, a method of suturing tissue of a portion of a stomach of a patient includes providing a suturing system with an imaging system capable of obtaining real-time video images; inserting the suturing system into the stomach through a natural orifice; overlaying virtual images over real-time video images on display; and suturing the portion of the stomach.

In some aspects, the virtual images include proposed suturing locations.

In some aspects, the virtual images include warning zones for non-suturing.

In some aspects, the virtual images include proposed suturing locations.

In some aspects, the virtual images include proposed suturing orientations.

In some aspects, the virtual images include anatomical landmarks.

In some aspects, the virtual images include fiducials.

In accordance with various principles of the present disclosure, an integrated needle and suture includes a needle having a sharp tissue piercing end and a shaft defining an opening; and a length of suture having a first end fixed to the needle and a second end, the suture having at least one barb along its length, the second end of the suture adapted to be drawn through the opening and self-retained through the opening once the at least one barb passes through the opening.

These and other features and advantages of the present disclosure, will be readily apparent from the following detailed description, the scope of the claimed invention being set out in the appended claims. While the following disclosure is presented in terms of aspects or embodiments, it should be appreciated that individual aspects can be claimed separately or in combination with aspects and features of that embodiment or any other embodiment.

### DETAILED DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

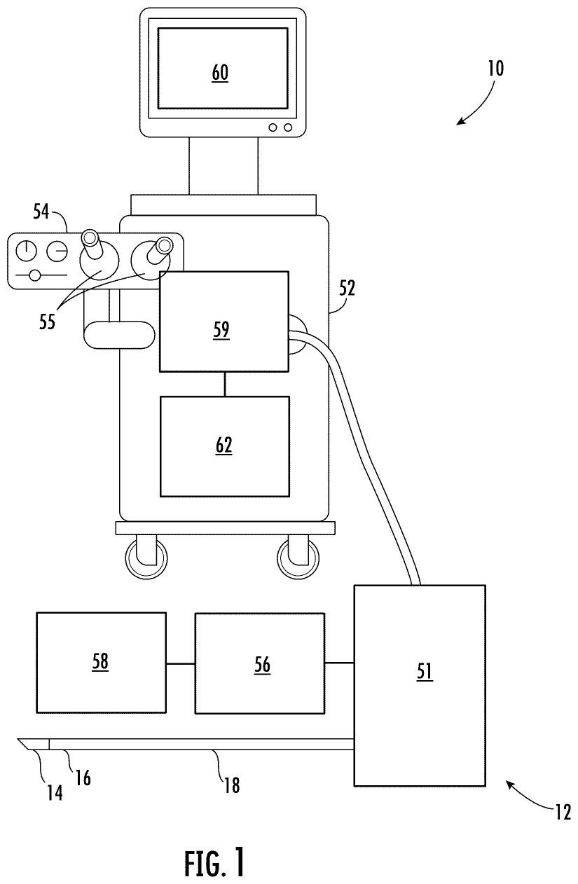

FIG. 1 is a general schematic diagram of a robotic suturing system.

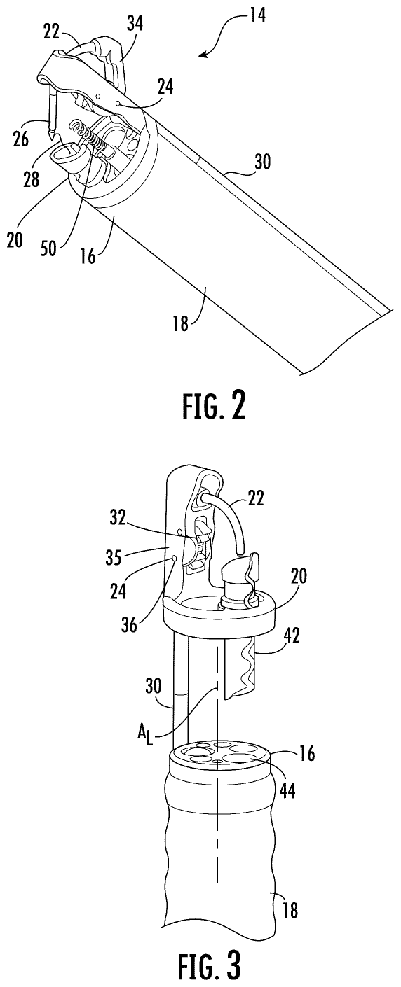

FIG. 2 is a perspective distal end view of an embodiment of the robotic suturing system.

FIG. 3 is an assembly view of the embodiment of the robotic suturing system of FIG. 2.

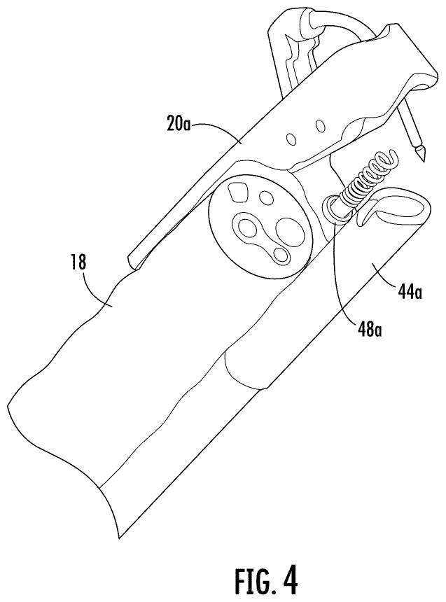

FIG. 4 is a perspective distal end view of another embodiment of the robotic suturing system.

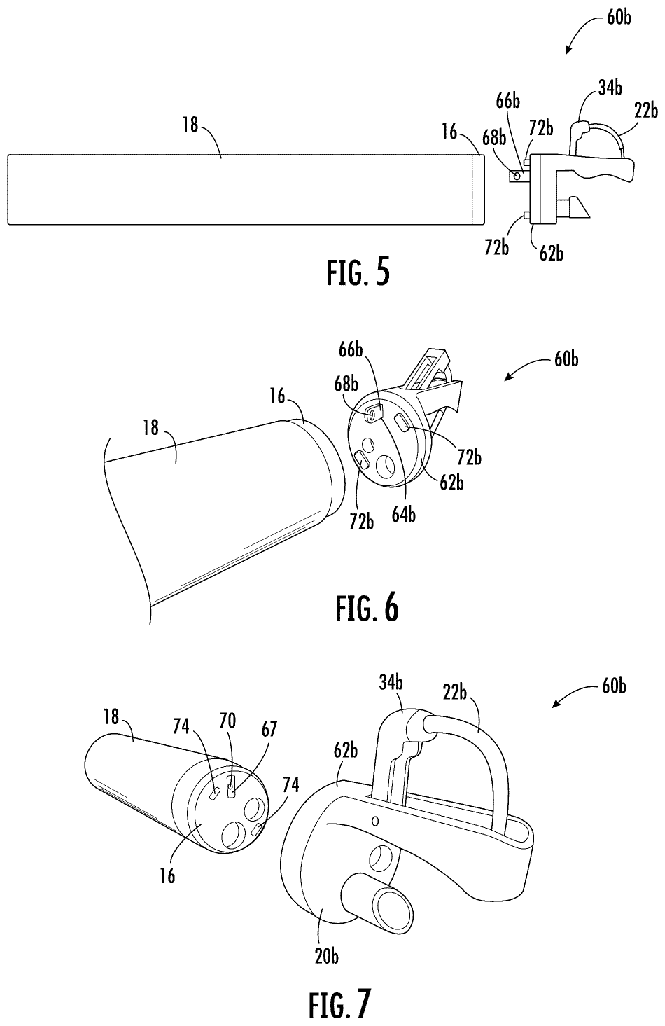

FIG. 5 is a schematic side elevation of a first cartridge end cap suturing system for the robotic suturing system.

FIG. 6 is a rear perspective view of the distal end of the system of FIG. 5.

FIG. 7 is a front perspective view of the distal end of the system of FIG. 5.

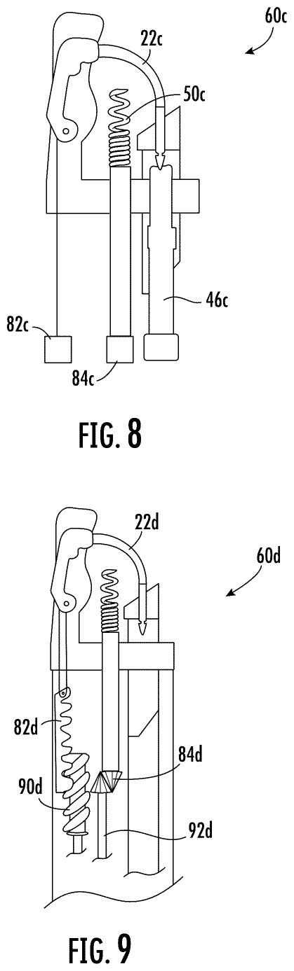

FIG. 8 is a schematic view of a second cartridge end cap suturing system for the robotic suturing system.

FIG. 9 is a schematic view of a third cartridge suturing system for the robotic suturing system.

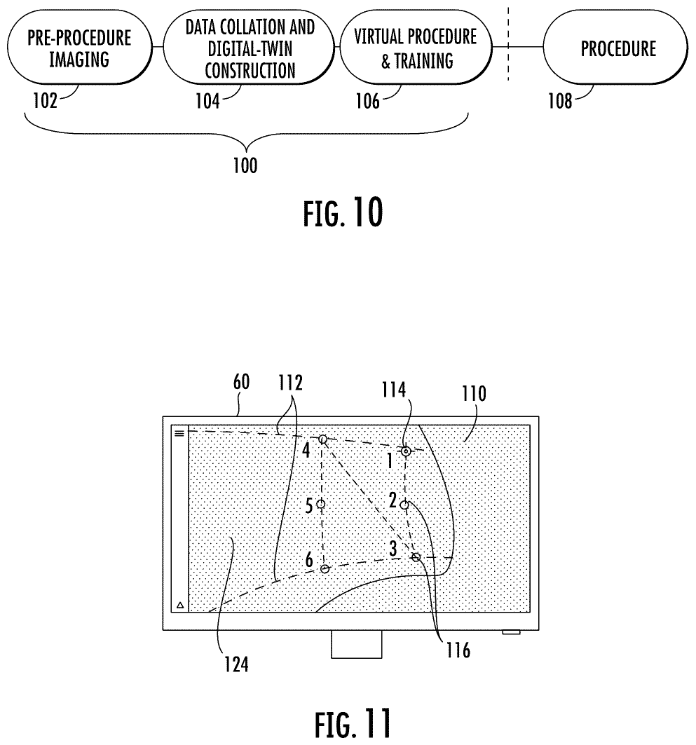

FIG. 10 is a flow chart of processes before and during a suturing procedure.

FIGS. 11, 12, 14-16, and 18-19 are schematic illustrations of the display of the robotic suturing system at various steps in a procedure and under various conditions.

FIGS. 13A-13C are distal end views of the robotic suturing system inside a gastrointestinal tract and illustrate orientating the suturing system relative to the tissue for optimal performance.

FIGS. 17A and 17B illustrate force feedback signals in ‘APPROVED’ and ‘WARNING’ conditions of bites through tissue.

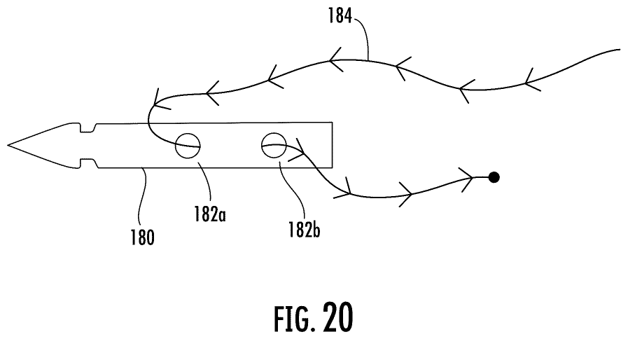

FIG. 20 is an embodiment of a needle and suture for use in suturing the gastrointestinal tract.

### DESCRIPTION OF THE INVENTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. In fact, it will be apparent to those skilled in the art that various modifications and variations can be made in the present disclosure without departing from the scope or spirit of the present subject matter. For instance, features illustrated or described as part of one embodiment can be used with another embodiment to yield a still further embodiment. Thus, it is intended that the present subject matter covers such modifications and variations as come within the scope of the appended claims and their equivalents.

It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device/system into a patient, or during positioning or delivery), and/or closest to a delivery device, and “distal” refers to the direction or location furthest from the user, such as when using the device (e.g., introducing the device into a patient, or during positioning or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a channel, a cavity, or a bore. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Various embodiments of devices, systems, and methods in accordance with various principles of the present disclosure will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

Turning now to FIGS. 1 and 2, a robotic suturing system 10 includes an endoluminal robot 12 and a suturing system 14 attached at a distal end 16 of the robot. The endoluminal robot 12 includes a robotic, shapeable insertion tube 18 advanceable into a natural orifice of a patient. The robotic system 12 is aware of the shape of the insertion tube and the location of the insertion tube in space.

Referring to FIGS. 2 and 3, the suturing system 14 includes an endcap 20 removably attached to the distal end 16 of the insertion tube 18. A needle holder arm 22 is mounted to the end cap 20 for rotation about an axis 24 and carries a removable needle 26 provided with a length of suture 28. The axis 24 is oriented transverse to the long axis A_{L} of the insertion tube 18.

In an embodiment, the suturing system includes a flexible transmission member 30, a gear train 32 coupled to a distal end portion of the transmission member 30, and a connecting member 34 pivotally coupled to a mounting bracket 35 at pivot pin 36 and extending to the needle holder arm 22. The removable needle 26 is connected to the needle holder arm 22 and is adapted to pierce tissue. When the transmission member 30 is actuated, the connecting member 34 moves the needle 26 and needle holder arm 22 in a direction to pierce tissue or in a direction to remove it from tissue. In one embodiment, the end cap 20 of the suturing system is operable substantially as described in U.S. Pat. No. 9,867,610, which is hereby incorporated herein in its entirety. (Appendix A)

In an embodiment, the end cap 20 of the suturing system is adapted to be removably mounted to the distal end 16 of the shapeable insertion tube 18. Any suitable manner of mounting the end cap to the insertion tube can be used. Referring to FIG. 3, the end cap 20 can be mounted over the distal end face 40 of the insertion tube 18 via a resilient partially tubular spring clamp 42 that extends proximally from the end cap and which inserts into a first instrument channel 44 of insertion tube 18. Outward force from the spring clamp 42 against a wall of the first instrument channel 44 secures the end cap 20 to the insertion tube 18 during use. The end cap 20 may alternatively be attached to the insertion tube via an external clamp that extends about a portion of the insertion tube, via a bayonet lock, via bands or tape, or any other suitable structure.

When mounted, one or more actuators from the robotic system 10 are coupled to the transmission member 30 such that, when activated, the robotic system can control the movement of the needle arm 22 and consequently the needle 26.

In an embodiment, the insertion tube 18 includes the first instrument channel 44 for passage of a needle exchange device (not shown) and preferably a second instrument channel 48 for passage of a tissue grasper 50 (FIG. 2). The needle exchange device is adapted to extend through the first instrument channel 44 and has a distal end adapted to receive, grasp, and release the needle 26 which would be located at the distal end of the first instrument channel. Exemplar needle exchange structure and operation is disclosed in detail in previously incorporated U.S. Pat. No. 9,867,610. Referring to FIG. 2, the illustrated example of an embodiment of a tissue grasper 50 includes a helical coil with a sharp distal tip that can be advanced and rotated into tissue to grasp the tissue, longitudinally retracted to retract the grasped tissue, and then counter-rotated to release the tissue. Such a tissue grasper 50 is disclosed in previously incorporated U.S. Pat. No. 9,867,610. Other tissue graspers, including but not limited to forceps and vacuum-assisted devices, may also be used.

While the above description of the flexible robotic surgical system suggests that the end cap 20 is substantially similar to that described in U.S. Pat. No. 9,867,610, it is appreciated that the robotic insertion tube 18 may include fewer or additional features than a conventional endoscope with two instrument channels, and the end cap constructions may be modified and coupled relative to the insertion tube in an appropriately mating manner. For example, referring to FIG. 4, one or both of the first and second instrument channels 44a and 48a for the needle exchange and tissue grasper operations and instruments may be provided external of the robotic insertion tube 18. By way of example, similar structure is described in detail in previously incorporated U.S. Pat. No. 11,051,800. The end cap 20a can be coupled in a suitable manner at and alongside the distal end of the robotic insertion tube as shown.

The above suturing systems 16 include an end cap 20 (20a) that is operable via a flexible transmission member 30 that extends along the outside of the robotic insertion tube 18 for operation from a proximal aspect thereof.

In other embodiments, the robotic suturing system can include an end cap integrated in a cartridge and which is adapted to interface to a suitably configured robotic insertion tube. Turning to FIGS. 5 through 7, by way of example, the end cap 20b can be integrated into a cartridge 60b. Cartridge 60b includes a base 62b for seating at the distal end 16 of the insertion tube 18, and a connecting member 34b extending from the needle holder arm 22b through an opening 64b in the base 62b. The connecting member 34b has a free end 66b, and structure at or adjacent its free end 66b for engaging with an actuator within the distal end 16 of the robotic insertion tube 18. In an embodiment, the free end 66b is adapted to fit into an opening 67 in the distal end 16 of the insertion tube. In an embodiment, the free end 66b has a hole 68b, and the distal end 16 of the insertion tube has a pin 70 which can be activated to advance crosswise through the opening 68b of the free end 66b to securely engage the free end 66b. The pin 70 is attached to a mechanism in the insertion tube 18 adapted to effect longitudinal displacement of the connecting member 34b and consequently movement of the needle holder arm 22b. The connecting member 34b can alternatively be configured in other ways for attachment to one or more actuators to effect longitudinal displacement thereof. The base 62b also includes tabs 72b adapted for insertion into recesses 74 at the distal end 16 of the insertion tube 18 and which provide for releasable attachment between the base 62b of the cartridge 60b and the distal end 16 of the insertion tube 18 in a manner that prevents rotation of the end cap 20b on the insertion tube 18. Other mechanism suitable to provide for releasable attachment between the cartridge 60b and insertion tube 18 can be used.

Turning to FIG. 8, another embodiment of a cartridge 60c is shown. The cartridge 60c includes a first connector 82c mechanically connected to the needle holder arm 22b and adapted to interface with a first actuator of the robotic insertion tube (not shown) at the distal end of the robotic insertion tube, and a rotatable tissue grasper 50c having a second connector 84c at its proximal end which is adapted to interface with a second actuator of the robotic insertion tube (not shown). The first connector 82c is adapted to permit transmission of longitudinal force. The first connector 82c can utilize, by way of example only, a bayonet connection, a threaded connection, a collet, rare earth magnet, an electro-magnet interface, a snap interface, a solenoid release pin, a dove tail interface, a keyway, a friction fit, a clip, a set screw, a gear interface and/or other suitable connectors to interface with a respective actuator on the robotic insertion tube. The second connector 84c is adapted to permit application of torque to the second connector from a respective actuator on the robotic insertion tube. The second connector 84c can utilize, by way of example, a collet, rare earth magnet, an electro-magnet interface, a snap interface, a solenoid release pin, a dove tail interface, a keyway, a friction fit, a c-clip, a set screw, a gear interface, and/or other suitable connectors. In addition, a needle exchange system 46c can similarly be provided in alignment with the distal end of the needle holder arm 22c when the arm is in a closed position and operated by a suitable connector integrated therewith and operated via the robotic insertion tube. As such, the cartridge 60c can integrate all the components that operate with the needle and on the tissue. After a surgical procedure the entire cartridge can be removed for cleaning, reprocessing, sterilization, and reuse. Alternatively, or additionally, after a determined number of surgical procedures, the cartridge can be discarded and replaced.

In the example of an embodiment illustrated in FIG. 9, the cartridge 60d has a first connector 82d in the form of a rack. The rack 82d is adapted to interface with a rotatable worm gear 90d operable from within the insertion tube 18d. As the worm gear 90d is rotated, the rack 82d is longitudinally displaced and effects opening and closing of the needle holder arm 22d. The second connector 84d includes a bevel gear to interface with a rotatable bevel gear 92d operable from within the insertion tube 18d.

Referring back to FIGS. 1 and 2, the robotic surgical system 10 also includes a mechanized system 51 to control the movement of the insertion tube 18 and the needle holder arm 22, via mechanical actuators, such as push-pull actuators and/or rotatable actuators or a gear drive mechanism like a worm gear. The robotic surgical system 10 includes a robot control system 52 to control movement of the mechanized system 51 based on inputs from the surgical system. A human interface 54 is coupled to the robot control system 10 which converts human manual input to movement of the insertion tube 18. The interface 54 can include, without limitation, joysticks 55, trackballs, a keyboard, buttons, knobs, haptic gloves or any other suitable interface to permit input from an operator. The robotic surgical system 10 also includes first sensors 56, including load cells and strain gages coupled to the actuators in the mechanized system 51, to monitor forces applied by the mechanized system and actuators. First sensors 56 may be located within the insertion tube or coupled via mechanical, optical, or electrical components to sensors external of the insertion tube. The robotic surgical system also includes a light source and camera 58, second sensors 59 to sense and identify the patient environment and optionally the needle and/or the suture, and a visual display 60 to display images from the camera optionally augmented by input from the first and second sensors and/or patient data. The robotic surgical system also includes a processing system 62 including a microprocessor that runs the robotic system software, a memory for storing software, an interface to access patient data, and which integrates the inputs from the subsystems together.

The robotic system 10 is adapted to map at least a portion of the gastrointestinal anatomy, and preferably at least the stomach, and generate a usable model thereof for use in pre-operative planning and/or real-time aspects of a procedure. Referring to FIG. 10, in accordance with an aspect of the present disclosure, the robotic surgical system permits mapping and imaging of the gastric environment in a planning step 100 before the surgical procedure. Patient imaging data, including, without limitation, weight and height, and/or patient scanning and visualization techniques, including, without limitation, MRI, CAT scanning, stereoscopic imaging, and/or other visualization and scanning techniques, is obtained at 102. Then a three-dimensional (3D) map of the shape of the patient's stomach is constructed at 104 based on the data and patient imaging data. The three-dimensional map is preferably a digital twin of the patient's anatomy. On the digital twin, the robotic system identifies and/or placement locations for suturing in the tissue, and the order for stitching based on the patient, patient orientation, procedure, and intended result. The physician can digitally review the proposed locations as well as the intended reshaped anatomy before performing the procedure on the display 60. A virtual procedure can be reviewed, including placement of the sutures and intended anatomical reconstruction, at 106. The proposed locations can be approved or modified. Then, the procedure can be performed 108.

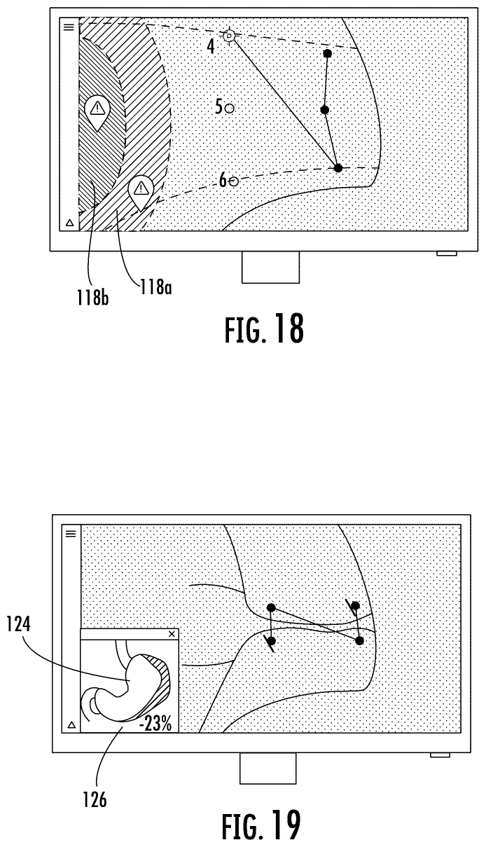

Referring to FIG. 11, during the procedure, the robotic system displays a real-time video image 110 as seen through the endoscopic camera. This image is combined with optional overlays of one or more of digitized or virtual images of the mapped anatomy, reference lines 112, a first recommended location 114 for stitching with the suture needle, subsequent recommended stitching locations 116 to effect the proposed stomach reconfiguration and the order placement of such stitches, warning zones 118a, 118b of tissue to avoid grabbing or stitching through (e.g., because such tissue overlies sensitive tissue) (described below with reference to FIG. 18). The overlay images are displayed on a display screen 60 and/or on augmented reality glasses. By way of example, system 10 can map and display a course of stiches, for example, projected stitches ‘1’ . . . ‘6’ to provide an intended stomach reduction, starting at highlighted proposed stitch ‘1’ at 114. Preferably, all overlay images can be turned ‘ON’ and ‘OFF’ as preferred by the operator.

In accordance with one aspect of the present disclosure, the robotic surgical system 10 is adapted to use real-time imaging acquisition from the optical system 58 and first and second sensors 56, 59 connected to the insertion tube 18. The optical system 58 and sensors 56, 59 can include, without limitation, stereo imaging optical sensors, laser scanning sensors, distance sensor, ultrasound sensors, CT scan devices, LIDAR sensors, or other sensors to display a real time shape of the stomach or other gastrointestinal tract structure on the display 60 as the structure is re-shaped via the suturing procedure; i.e., when the tissue is reshaped through the tensioning of sutures and restraining of the sutures via cinching, tying off, etc. As the gastrointestinal tract 124 is re-shaped, the system 10 displays updated proposed locations for tissues sutures, patterns for the suture, a proposed number of ideal sutures for a proposed procedure and stomach reduction, sensitive structure locations, stomach sizing information like residual volume, volume reduction 126 (FIG. 19), expected remaining procedure time, and intraluminal pressure due to current gas insufflation. Other useful parameters can be displayed as well. Thus, the system assists the operator to navigate and maintain optimal technique as the anatomy is reconfigured during intraluminal suturing to optimize outcomes, reduce procedure time, reduce the risk of suture entanglement or other issues that could require troubleshooting and may delay the procedure, and reduce the amount of training required to master a certain procedure. By way of example, during an endoluminal sleeve gastroplasty procedure, the stomach is reconfigured into a narrower and shorter shape which can be disorienting for a human operator but can be managed by the robot visualization system to guide the operator.

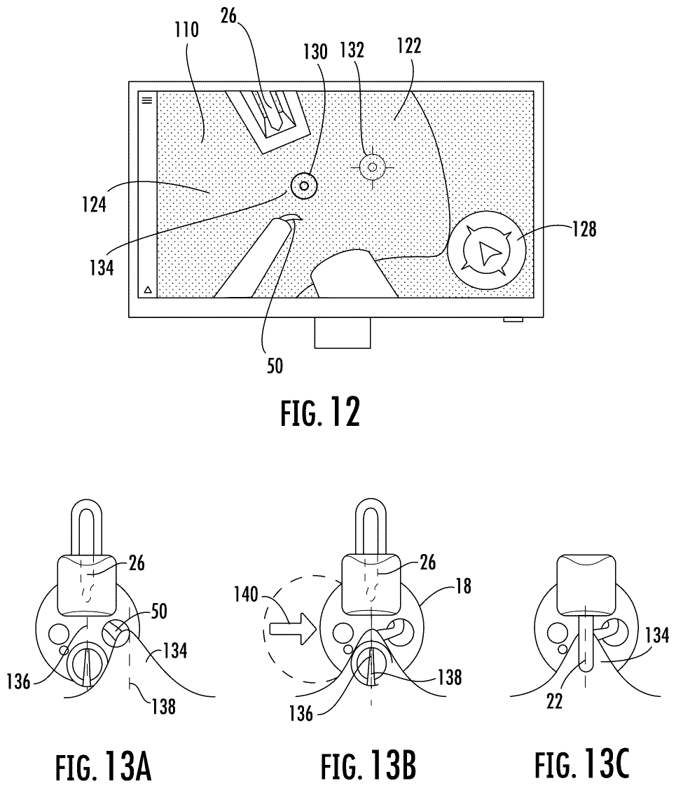

Turning now to FIG. 12, the real-time imaging system identifies landmarks on the stomach 124 or other portions of the gastrointestinal tract, even as at least a portion of the gastrointestinal tract is reconfigured. A visualized compass 128 and virtual markers of key anatomical locations, e.g., posterior wall, anterior wall, fundus, pylorus, assist the operator. In addition, the robotic system can be designed to deploy or display fiducials in any form, including, but limited to, physical markers, such as dye markers sprayed onto tissue, laser or argon plasma burned markers on the tissue, colored or patterned physical marks attached to the tissue, radiopaque markers attached to the tissue, active signal-generating markers, or virtual indicia. Physical fiducials can be sensed by the image sensors or other sensors at the distal end of the insertion tube to facilitate sensing the shape of a portion of the gastrointestinal tract as the portion of the tract is reconfigured. Virtual fiducial 130 can indicate the location at which the system is currently directed to pass the suture needle, while another fiducial 132 can indicate the preferred location to pass the suture needle for a full thickness pass through the tissue. This facilitates orienting the operator during the procedure. Virtual markers can be overlaid on camera images 110 of the stomach anatomy 124 and used to guide a human operator to move the end of the insertion tube relative to tissue so that the suture needle extends through the grasped tissue 134, an appropriate amount (full thickness) of tissue, and at an intended orientation relative to the grasped tissue. Alternatively, overlaid information to augment the real-time image can also be transmitted and displayed on a special headset or glasses that the system operator can wear during the procedure when system input and guidance is desired.

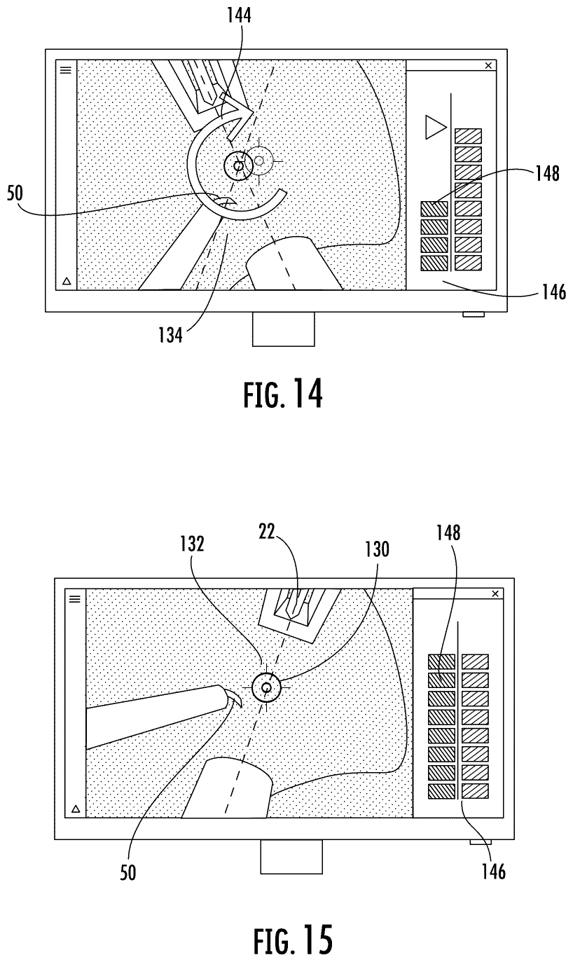

Still referring to FIGS. 12, the illustrated example of an embodiment of a system is shown with the tissue grasper 50 engaged in the stomach tissue 134. However, as indicated by the offset locations of fiducials 130, 132, the grasped tissue 134 is not optimally located so that the needle 26 will extend through a full thickness of the tissue, as preferred and discussed below. This is also represented at FIG. 13A, which illustrates that the needle pathway 136 is laterally offset from a path 138 which extends through a full thickness of the grasped tissue 134. In order for the suture needle 26 to take a full thickness pass through the tissue (along path 138), the path 136 of the suture needle 26 must be laterally displaced toward the marker 132. The controls 54 operably connected to the robotic insertion tube 18 are manipulated to move the insertion tube in the direction of arrow 140 and align the needle pathway 136 with full thickness pathway 138, as shown in FIG. 13B. In addition, as shown at FIG. 14, the robotic system monitors the rotational orientation of the suturing system and can guide the operator by marker at 144, or actively rotate the end cap and suture needle as necessary, to provide a path for the needle 26 so that the tissue is optimally positioned to be sutured and/or the suture is optimally arranged to extend between suture locations. The intended tissue should be at the center of the needle trajectory as the needle is rotated about its rotation axis on the needle holder arm. Image analysis is used to detect tissue within a field of view of the optical sensors and detect and confirm that the tissue is at an appropriately centered and distanced tissue location. In addition, the system identifies whether the end cap 20 supporting the needle 22 should be rotated to provide an optimal needle trajectory through the tissue to maximize tissue thickness by the needle, as shown in FIG. 14. Once the system and operator determine that the needle 26 is appropriately oriented relative to the tissue, the system can be activated to advance the needle 26 through the retracted tissue 134. The system may include a lockout, with override function, to prevent unintentionally advancing the needle 26 into tissue unless the needle is oriented in a desirable manner.

The insertion tube 18 may optionally include endoscopic ultrasound (EUS) capability. Such capability may extend to the distal end of the suturing system. Such endoscopic ultrasound capability enables the operator to visualize anatomic structure outside of the gastrointestinal tract during intraluminal suturing. This permits the location and monitoring of sensitive structures outside the gastrointestinal tract and mitigates the risk of suturing through the gastrointestinal tract and unintentionally engaging external anatomy. By way of example, the endoscopic ultrasound can provide feedback to optimize penetration depth of the tissue grasper or the needle (or another fastener). Fastener depth, and the extent of tissue retraction by the tissue grasper 50, may be optimized based on location and potential underlying tissues and organs. Ultrasound readings may also be used to optimize gastrointestinal tract insufflation based on tissue thickness, with thinner tissues being insufflated to higher pressures, and thicker tissues insufflated to lower pressures. Ultrasound can also be used to verify whether the suture needle 26 has extended through a full thickness of tissue.

In another aspect of the system, the system analyzes the first sensors 56 coupled to the tissue grasper 50 to obtain a force ‘fingerprint’ to verify that the grasper 50 has properly engaged or moved through tissue. The system utilizes force feedback from torque gauges coupled to the actuators of the tissue grasper 50 to determine when tissue has been properly engaged. When, for example, the helical coil tissue grasper 50 is rotated in a first direction (e.g., clockwise), the sensed torque is related to the tissue depth engaged by the coil. The system includes a feedback function optimized to engage the tissue until a determined torque is sensed to ensure consistent tissue engagement depth, and optimal retention force between the grasper and tissue. Similarly, in another function, if the torque sensed drops and then subsequently increases, the system can be set to identify that as the coil tissue grasper rotating through the stomach wall and then engaging structure external of the stomach, which would trigger a warning condition.

In addition, the first sensors 56, 59 are used to identify if unintended structures are engaged external the stomach wall. For example, when retracting the tissue grasper instrument with grasped stomach wall, there will be an expected sensed force on the tissue grasper. If a significantly higher force is sensed, such can indicate that the tissue grasper grasped an organ or other tissue outside the stomach. A warning or alarm indicator, with appropriate identifier of the trigger, can also be presented under such conditions. In such, case the tissue should be released and re-grasped at a more appropriate depth and/or anatomical location.

Further, the sensors 56 may be coupled to the tissue grasper 50 and operated to indicate on the display at 146 the force to which the grasped stomach tissue 134 is subject. FIG. 14 provides an example of an embodiment of a force meter display 146 with an indication of lower force applied by the tissue grasper at 148 (which may indicate a failure to provide full thickness suturing), whereas FIG. 15 illustrates larger force by the tissue grasper 50 on the meter at 148, presumably indicating the grasper 50 has grasped and retracted the stomach tissue back for a full thickness suture pass. In addition, FIG. 15 shows the virtual markers 130, 132 for the passage of the suture needle and the recommended suture path 132 in alignment. As such, FIG. 15 illustrates an ideal condition prior to advancing the needle 26 through the tissue 134. Thus, the system is ready to advance the suture needle 26 through the retracted tissue 134.

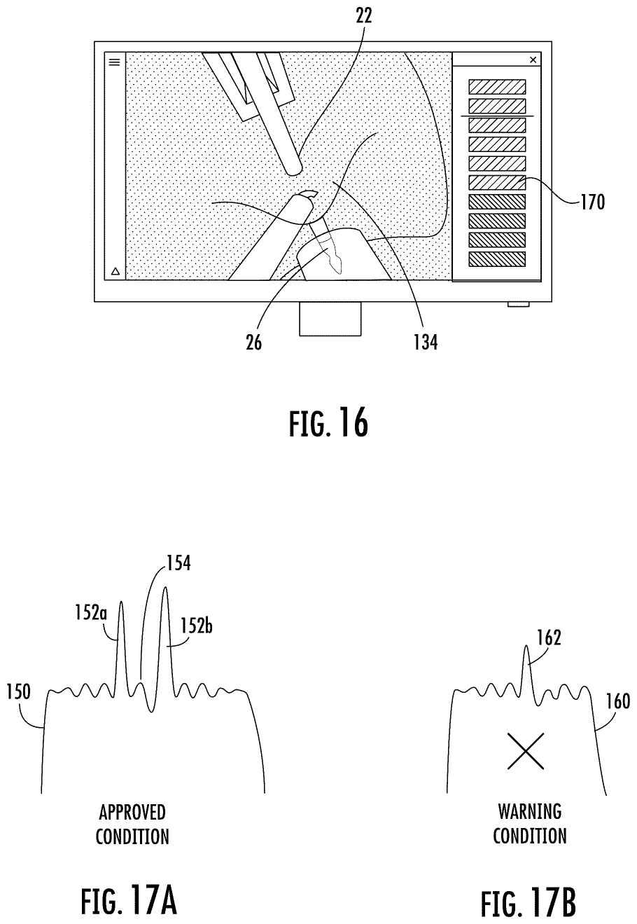

Turning to FIGS. 13C and 16, the system 10 is then operated to move the needle 26 through the retracted tissue 134, and preferably through a full thickness of tissue. The system can use several feedback mechanisms to confirm such full thickness tissue suturing. In one aspect, the system uses force feedback from strain gauges attached to the needle holder arm 22 or strain gauge/load cell suspended pulleys or linear sliders that drive the needle holder arm actuation cable to confirm that the suture needle has moved through the intended thickness of tissue. When performing endoluminal sleeve gastrectomy, it is preferred that two portions of the stomach wall be sutured together through the full thickness of the stomach wall; i.e., from the inside wall of a first portion of the stomach to the outside wall of the first portion, and then from the outside of the second portion of the stomach to the inside wall of the second portion in order to ensure the long term stability of the tissue approximation. This is in contrast to partial thickness suturing where the needle and suture pass within the stomach wall and not full out and back through. The intended full thickness bite of the suture path is verified in real time by monitoring the forces acting on the tissue penetrating part of the suture mechanism by means of force sensors. The monitored forces will be characteristic of the needle interaction with different soft tissue types like the mucosa, submucosa, muscle, and serosa due to differences in penetration, dilation, and frictional forces. Referring to FIG. 17A, in a full thickness needle path through tissue, the first sensors 56 will identify a signal 150 having two distinct peaks 152a. 152b and a distinguishable drop 154 between the peaks 152a, 152b indicating the needle 26 passing through two separate serosas as the needle traverses out through the first stomach portion, and back through the second stomach portion. This signal indicates a full thickness stitch by the needle with high confidence of durability. In distinction, referring to FIG. 17B, a differently shaped force signal 160 with only a single peak 162 is shown. The system can identify the distinction and cause an approval indicator to be presented to the operator when signal 150 is identified and/or a warning or alarm indicator to be provided to the operator when signal 160 is identified. Under the conditions of a warning event, the system 10 may recommend that a duplicate suture bite should be repeated through the tissues portions. The display 60 may include digital gauges 170 and displays for warnings to operator, monitoring the patient, and recommending subsequent stages of the procedure that update and change depending on the stage of the procedure and the active component (FIG. 16). Such force monitoring may also be used to detect inadvertent puncturing of structures external to the gastrointestinal tract during the intraluminal suturing and subsequent retraction of the suture needle to prevent outside structures to be connected to the outside of the gastrointestinal tract during full thickness suturing.

The system 10 includes a pressure gauge at one of the insertion tube or suturing system to maintain optimal insufflation of the gastrointestinal space. If pressure is below a first predetermined level, the system automatically operates a valve or pump to add insufflation gas through a port in the insertion tube. If the pressure is above a second predetermined level, the system automatically operates a vent or actively pumps out insufflation fluid from the gastrointestinal space. The level of insufflation may be automatically adjusted by the robotic control system to ensure continuous optimal insufflation levels for visualization with consideration to which part of the tissue approximation sequence the robotic system maybe in, for example, higher level of insufflation for initial visualization of the working space prior to the procedure and for initial space mapping by the robotic system, lower insufflation during tissue acquisition to limit tissue grasper penetration depth and to reduce the risk of capturing outside structures, and lower insufflation during final tissue approximation to reduce forces on the suture and tissue.

Turning now to FIG. 18, the system displays a later progress, after stitches ‘1’, ‘2’, and ‘3’ have been made, and an approach to stitch ‘4’ is next recommended. In addition, as referenced above, the system identifies areas 118a, 118b, e.g., near the fundus, overlaid with ‘warning’ symbols. One area 118a may be displayed in a first color and indicate a first type of warning (a first cautionary tissue thickness), whereas a second area 118b may be displayed in a second color and indicate a second type of warning (an underlying organ and/or a second cautionary tissue thickness).

Once the suture has been passed through the intended areas, the suture is tensioned to draw the sutured portions of the stomach (or other gastrointestinal system) toward each other. The tissue may be drawn into apposition causing the various regions to contact each other, or may otherwise be drawn to move and relocate the stomach regions depending on the intended procedure and results. Referring to FIG. 19, after the suture is appropriately tensioned, the suture is cinched by applying a cinch device, tying off, using an integrated securing element, or via other device or method. The system shows the current modification in shape to the stomach at 124, including the reduction in capacity at 126. Additional sutures can be applied as necessary to complete the procedure.

In accordance with aspect of the present disclosure, the suture can be managed with the robotic system. In management, the suture can be better visualized, accessed, controlled and manipulated. With respect to visualization within the anatomical space, the length of suture attached to the suture needle is marked with aids to facilitate robotic suturing. In an embodiment, the suture can be a high contrast color, and the display system is color filtered to enhance visualization of the suture in the surgical field. Using a distinct suture color with high contrast, the suture is readily differentiated by the robotic system from other parts of the endoluminal suturing system, from tissue, and from blood. In addition, in an embodiment, the suture includes a fluorescent dye and the illumination wavelength of the robotic imaging system is adjusted to the dye frequency to visualize and locate all sutures in the gastric tract. In an embodiment, the suture is radiopaque to facilitate visualization of the suture during and/or after completion of a surgical procedure under fluoroscopy. In this manner, the durability of the sutures can be determined over time. In an embodiment, the suture has a surface texture and/or structure that has high contrast under ultrasound imaging, for example, by having a dimpled surface, and the location of the suture can automatically be detected by the system. The suture material may also be chosen to give a high ultrasound signal strength. In an embodiment, the suture is provided with a surface pattern that is recognizable by the robotic system to indicate the end of the suture attached to the needle, a free end of the suture, and locations therebetween. Such pattern or patterns can be similar to a bar code that can be visually read by the robotic system to identify exactly which portion of the suture is currently under direct visualization and to know whether portions of the suture are crossed-over in an undesirable manner. Managing the path and location of the needle is a valuable advantage of a robotic system. In an embodiment the robotic system can use the visual information from the suture to confirm the amount and correct location of stitches placed and length of suture used and use such data for example to update a computer-generated rendered representation of the procedural space and to update the recommended suture locations indicated on or superimposed on the real time image the operator sees.

In another aspect of management, the tension on the suture is controlled. A predetermined suture tension (within limits) or slack is actively maintained on the suture throughout the procedure. Optimal slack allows for running the stitches in the tissue, maneuverability of the suturing system, and reduces suture drag during stitching. However, there should not be so much slack as to prevent optimization of suture tensioning during final tissue approximation or to increase the likelihood of suture entanglement. The ideal tension during final tissue approximation is below a tensile strength of a knot tied in the suture or the strength of suture to mitigate breakage of the suture as well as below the force required to rip the suture through tissue, yet high enough to obtain suitable tissue apposition. In addition, it is desirable to maintain slight tension on the suture when passing instruments through the instrument channel with the suture, as loose suture can wrap around the instruments and bind them. To provide the appropriate tension on the suture, the suture may be fed through mechanisms such as a roller system, a pulley, or over a spool mounted to a strain gauge. When tension is too high, the roller, pulley or spool can then unwind or be moved to release more suture into the surgical field. Then, when tension is too low, the roller, pulley or spool can wind or move to take up suture from the surgical field and increase tension. The process can be manual or automatically controlled based on preset parameters.

In another aspect of management, the system monitors and prevents sutures from crossing over one another in a manner that could cause inadvertent clamping, twisting, or knotting of the suture, unnecessary drag on the suture, entanglement. The suture position is monitored to detect cross-over and provide a warning signal to an operator that a cross-over situation has occurred, or the current suture position is susceptible to cross-over and automatically suggest or adjust movement of the robotic system in a manner that prevents suture crossover. For example, the end cap 20 can be automatically rotated by the insertion tube 18 or the insertion tube 18 can be rotated to mitigate suture cross-over.

In another aspect of suture management, overall usage of the endoscopic suturing procedure is reduced in complexity by way of the robotic platform. In one example, in a manually operable endoscopic suturing system similarly structured to the robotic suturing system, the suture needle is advanced through the tissue and then the trailing end is tensioned and terminated, for example with a cinch, knot, or crimp. However, in the robotic system, the trailing end of the suture can be utilized in different ways. For example, once suturing of the tissue is completed, the trailing end of the suture can be terminated and extra suture cut and removed, and then the needle side of the suture can be tensioned, terminated, and cut. Further, the trailing end of the short suture for a procedure can be preinstalled with a permanent anchor such as a T-Tag in the form of a pivoting bar, polypropylene disc, or curled nickel-titanium suture tail, and which does not require termination and cutting after the suturing.

In an example of an embodiment illustrated in FIG. 20, a needle 180 with one or more openings 182a, 182b can be used which interlocks with a barbed suture 184 to create a one-way self-locking tensioning mechanism. As tension is applied to the suture 184, the suture is drawn relative to the needle 180 and the tissue is approximated from the needle side in a locking manner. The remainder of the barbed suture 184 is then cut from the locked needle 180 and suture 184. As may be appreciated with reference to FIG. 20, the barbs of the barbed suture 184 may be angled with respect to the main body of the suture to allow the barbs to pass through the one or more openings 182a, 182b of the needle 180 in one direction, but not in the opposite direction, thereby being locked against movement in such opposite direction.

In another aspect of suture management, the suturing can be performed in a direction different from that which occurs in a manually operable endoscopic suturing system similarly structured to the robotic suturing system. In the manually operable system, the suturing is performed in a right to left direction, or top to bottom direction, or distal to proximal direction. This is intended to keep the suture in the field of view and prevent suture entanglement or crossing. However, the robotic system with its enhanced suture awareness and maneuverability capabilities enables suturing in a more natural left to right, or bottom to top, or proximal to distal direction, and removes the need for the operator to carefully pre-plan the suturing path and pattern. The robotic system is programmed to analyze the anatomy of the surgical field, identify an optimal suture pattern for a given procedure in view of the anatomy, highlight an optimal suture pattern for the surgical procedure, and highlight an optimal stitching direction for the suture pattern. The highlighted pattern and directions are preferably displayed on a real-time image of the surgical field as imaged by the optical sensor using, for example, augmented reality, and/or a virtual three-dimensional image constructed of the anatomy of the surgical field constructed from real-time or previously obtained imaging data.

There have been described and illustrated herein embodiments of robotic suturing systems for bariatric treatments, and methods of performing robotic suturing and bariatric treatments under robotic assistance. Although embodiments of the present disclosure may be described with specific reference to medical devices and systems and procedures for treating the gastrointestinal system, it should be appreciated that such medical devices, systems, and methods may be used to treat tissues of the abdominal cavity, digestive system, urinary tract, reproductive tract, respiratory system, cardiovascular system, circulatory system, and the like. Moreover, while particular embodiments of the inventions have been described, it is not intended that the inventions be limited thereto, as it is intended that the inventions be as broad in scope as the art will allow and that the specification be read likewise. It will therefore be appreciated by those skilled in the art that yet other modifications could be made to the provided invention without deviating from its scope as claimed. Thus, references to elements or structures or features in the drawings must be appreciated as references to examples of embodiments of the disclosure, and should not be understood as limiting the disclosure to the specific elements, structures, or features illustrated. Other examples of manners of implementing the disclosed principles will occur to a person of ordinary skill in the art upon reading this disclosure. It should be apparent to those of ordinary skill in the art that variations can be applied to the disclosed devices, systems, and/or methods, and/or to the sequence of steps of the method described herein without departing from the concept, spirit, and scope of the disclosure. It will be appreciated that various features described with respect to one embodiment typically may be applied to another embodiment, whether or not explicitly indicated, and either singly or in any combination thereof. Moreover, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A robotic suturing system for use in a gastrointestinal tract of a human patient, the human patient having a natural orifice in communication with the gastrointestinal tract, comprising:
a flexible, shapeable endoluminal robot adapted to be inserted into the gastrointestinal tract through the natural orifice, the endoluminal robot having a proximal end and a distal end;
a suturing tool removably coupled to the distal end of the flexible endoluminal robot, the suturing tool adapted to grasp tissue and advance a needle with suture through the grasped tissue;
a robot control system configured to control movement of the endoluminal robot and the needle on the suturing tool;
an imaging system for capturing real-time images of the gastrointestinal tract; and
a display for presenting the real-time images to an operator.

2. The robotic suturing system of claim 1, wherein the robot control system includes actuators to operate the endoluminal robot and the suturing tool, and an operator interface to control the actuators.

3. The robotic suturing system of claim 1, further comprising sensors adapted to map at least a portion of the gastrointestinal tract.

4. The robotic suturing system of claim 1, wherein the imaging system includes at least one of optical sensors, laser scanning sensors, distance sensors, ultrasound sensors, CT scan devices, and LIDAR sensors.

5. The robotic suturing system of claim 1, further comprising a mapping system to define a virtual map of a portion of the gastrointestinal tract.

6. The robotic suturing system of claim 5, further comprising a modelling system to propose pre-operative planning of a procedure in a gastrointestinal tract.

7. The robotic suturing system of claim 5, wherein the modelling system is adapted to project aspects of the preoperative planning over the real-time images.

8. The robotic suturing system of claim 1, further comprising at least one sensor adapted to determine whether the needle is inserted through a full thickness of the tissue.

9. The robotic suturing system of claim 1, wherein the display is also adapted to present virtual images to guide the operator.

10. The robotic suturing system of claim 9, wherein the virtual images include at least one of: proposed suturing locations, warning zones for non-suturing, proposed suturing locations, proposed suturing orientations, anatomical landmarks, or fiducials.

11. A method of suturing tissue of a portion of a gastrointestinal tract of a patient, comprising:
mapping at least a portion of the gastrointestinal tract;
obtaining imaging data of the patient;
constructing a digital twin of the portion of the gastrointestinal tract;
identifying proposed locations for suturing in the digital twin;
providing a suturing system with an imaging system capable of obtaining real-time video images;
inserting the suturing system into the gastrointestinal tract through a natural orifice;
virtually overlaying the proposed locations over real-time video images; and
suturing the portion of the gastrointestinal system.

12. The method of claim 11, further comprising suturing at the proposed locations.

13. The method of claim 11, further comprising updating the proposed locations for suturing as the portion of the gastrointestinal tract is sutured.

14. The method of claim 11, further comprising providing virtual overlays of at least one of warning zones, and estimated current volume reduction of the portion of the gastrointestinal tract affected by the suturing, anatomical landmarks, over real-time video images.

15. The method of claim 14, further comprising updating the virtual overlays as the gastrointestinal tract is sutured.

16. The method of claim 11, further comprising providing fiducials on landmarks of the gastrointestinal tract to guide a human operator.

17. The method of claim 11, further comprising monitoring whether the needle passes through a full thickness of the tissue.

18. The method of claim 11, further comprising:
before suturing, grasping tissue;
monitoring a force on the grasped tissue; and
releasing the grasped tissue if the force exceeds a threshold.

19. The method of claim 11, further comprising monitoring a tension on the suture.

20. An integrated needle and suture, comprising:
a needle including a sharp tissue piercing end and a shaft defining an opening; and
a length of suture having a first end fixed to the needle and a second end, the suture having at least one barb along its length;
wherein the second end of the suture is adapted to be drawn through the opening and self-retained through the opening once the at least one barb passes through the opening.
